(19) United States
(12) Patent Application Publication
(10) Pub. No.: US 2025/0258101 A1
(43) Pub. Date: Aug. 14, 2025

(54) FRONTSCATTERING REFLECTOR FOR RAMAN SPECTROSCOPY

(71) Applicant: Thermo Scientific Portable Analytical Instruments Inc., Tewksbury, MA (US)

(72) Inventor: Audrey Brand, Tewksbury, MA (US)

(21) Appl. No.: 19/048,294

(22) Filed: Feb. 7, 2025

Related U.S. Application Data
(60) Provisional application No. 63/551,709, filed on Feb. 9, 2024.

(51) Int. Cl. G01N 21/65

(52) U.S. Cl. CPC G01N 21/65; G01N 2201/0636

(57) ABSTRACT

Systems and methods for conducting spectroscopic imaging, such as Raman spectroscopy. One example provides an optical analysis system includes a light source generating an excitation light and an attachment. The attachment includes a reflective surface positioned to reflect light from a sample toward a probe of the optical analysis system. The attachment includes a holder supporting the reflective surface, wherein the sample is positioned between the probe and the reflective surface.

### RELATED APPLICATIONS

This application claims priority to U.S. Provisional Application No. 63/551,709, filed Feb. 9, 2024, the entire content of which is incorporated by reference herein.

### FIELD

Examples described herein generally relates to systems and methods for conducting optical material analysis, such as Raman spectroscopy.

### BACKGROUND

Raman spectroscopy is an effective tool for identifying and characterizing various sample compounds and substances. In Raman spectroscopy, light, typically from a laser and of a known wavelength, is directed at a sample compound or substance (referred to herein as a “sample”). The laser photons (also sometimes referred to as a Raman pump) inelastically scatter, or “Raman scatter,” off the molecules in the sample and experience wavelength shifting to new frequencies given by bond vibrational frequencies present in the molecules of the sample. The precise nature of this wavelength shifting depends upon the materials present in the sample. A unique wavelength signature (typically called the Raman signature) is produced by each sample. This unique Raman signature permits the sample to be identified and characterized. More specifically, the spectrum of light returning from the sample is analyzed with a spectrometer to identify the Raman-induced wavelength shifting from the Raman pump light, and this wavelength signature is compared (e.g., by a computing device) with a library of known Raman signatures to identify characteristics of the sample.

### SUMMARY

A probe that includes or is coupled with a light source and a detector may be used to direct light at a sample and detect light. In Raman spectroscopy, when light is directed at a sample, the resulting Raman scatter (henceforth referred to as a Raman signal) occurs in all directions. Accordingly, only Raman signal that is scattered toward the probe (e.g., backscattered light) is detected by the detector. While backscattered Raman signals provide value information about the sample, in some instances, the signal is too weak for analysis by a spectrometer. For example, particularly small samples may produce too little Raman scatter for analysis by the spectrometer. Further, some samples produce less Raman scatter due to their chemical composition.

Examples described herein capture both the backscattered and frontscattered light. Capturing frontscattered light, or Raman signal scattered away from the probe (or in, general, the light detector or light path associated with such a detector) (e.g., separately or in combination with backscattered light) may allow a spectrometer to identify additional insights into the sample environment, such as distribution and composition of sample cells, media, and other components of the sample or environment. By capturing frontscattered light by the probe, spectrometers and other instruments described herein receive a boosted Raman signal when compared to configurations that only capture backscattered light. Capturing a boosted Raman signal allows for a more accurate analysis of the sample and the sample environment, including the identification and quantification of various biomolecules, metabolites, and other sample parameters. Additionally, a boosted Raman signal provides for improved process control, better product quality, and enhanced efficiency in biopharmaceutical manufacturing for users of the spectrometer.

In one aspect, an optical analysis system includes a light source generating an excitation light, a light analyzer, a reflective surface, and an optical lens. The optical lens is configured to direct the excitation light to a sample at a focal region. The focal region may be from an effective focal length from the optical lens. The sample is positioned between the optical lens and the reflective surface. The optical lens is configured to receive backscattered light from the sample, direct the backscattered light to the light analyzer. The optical lens also receives frontscattered light from the sample, which is reflected from the reflective surface and directed to the light analyzer.

In another aspect, an attachment for an optical analysis system includes a reflective surface positioned to reflect light frontscattered from a sample toward the optical analysis system. The optical analysis system may include a probe for emitting a light toward a sample and collecting light scattered from the sample. The attachment may include a holder supporting the reflective surface. The holder may be coupled to the optical analysis system such that the light emitted from the focal region of the excitation light is positioned between the probe and the reflective surface. The reflective surface reflects light scattered away from the sample back toward the probe.

The reflective surface may have a curved surface. For example, the reflective surface may be a concave mirror, a spherical mirror, a parabolic mirror, or the like. In some aspects, the reflective surface may have other shapes, such as, for example, a plurality of flat mirrors arranged in a curved (e.g., a parabolic) manner.

The attachment may be coupled to the optical analysis system (or one or more portions thereof) in various ways and, in some aspects, may be removably couplable to the optical analysis system (or one or more portions thereof). For example, in some aspects, the attachment may include an attaching portion and the holder is connected to the attaching portion via one or more support structures (e.g., one or more legs, beams, prongs, tines, etc.). The attaching portion may couple the attachment to a probe (e.g., a probe head) of the optical analysis system, a housing of the optical analysis system, or a combination thereof. The attaching portion may include a channel in which the probe head slides into the attaching portion such that the attaching portion 402 surrounds a portion of the housing of the probe. The attachment may also include a fastening portion (e.g., a screw) for securing the attachment to the optical analysis system (or one or more portions thereof). The attachment may be removable coupled to the optical analysis system (or one or more portions thereof) or may be formed with the optical analysis system (or one or more portions thereof) such that the attachment is not configured to be removed (e.g., through welding, bonding, fusing, or otherwise fastening the attachment (e.g., the one or more support structures) to optical analysis system such that the components are not removable).

The support structures may position the holder (i.e., the reflective surface support by the holder) at a distance from the attaching portion. The support structures may define one or more apertures that allow flow or passage of the sample. Alternatively or in addition, the one or more apertures are configured to receive a vessel containing the sample. Also, one or more of the support structures may support a vessel containing the sample.

In some aspects, the attachment may include a housing, wherein portions of the housing may function as the holder, the one or more support surfaces, the attaching portion, or a combination thereof. For example, the attachment may include a housing that forms a chamber in which the sample is situated. The housing may be configured to interface with a fluid inlet and a fluid outlet, which allows the sample to enter and exit the chamber formed by the housing. A first portion of the housing is configured to receive (e.g., removably or permanently) a probe of the optical analysis system (e.g., a probe head), and a second portion of the housing functions as the holder and supports the reflective surface. Portions of the housing may function as the one or more support structures that position the reflective surface to face the probe when received into the housing.

There is no specific requirement that a system, method, or technique relating to Raman spectroscopy include all of the details characterized herein to obtain some benefit according to the present disclosure. Thus, the specific examples characterized herein are meant to be example applications of the techniques described and alternatives are possible.

### BRIEF DESCRIPTION OF THE DRAWINGS

Features and advantages of the present technology will become more apparent from the following detailed description of examples thereof taken in conjunction with the accompanying drawings in which:

FIG. 1 is a perspective view of an example spectroscopy system in use with a liquid sample container.

FIG. 2 is a block diagram of the spectroscopy system of FIG. 1.

FIG. 3 is a block diagram of an example controller component.

FIG. 4 is a side view of an example Raman probe and an optical attachment submersed in a sample.

FIG. 5A is a perspective view of the optical attachment of FIG. 4.

FIG. 5B is a side view of the optical attachment of FIG. 4.

FIG. 5C is a top view of the optical attachment of FIG. 4.

FIG. 6A, FIG. 6B, FIG. 6C, and FIG. 6D are side views of an optical system at various steps of retrieval of light scattered from a sample.

FIG. 7A is a perspective view of an example Raman probe and an optical attachment supporting a vial.

FIG. 7B shows data acquired with and without an attachment.

FIG. 8 is a side view of another example Raman probe and an optical attachment supporting a vial.

FIG. 9 is a side view of another example optical attachment coupled to a Raman probe.

FIG. 10A is a side view of the example Raman probe and optical attachment of FIG. 4 submerged in a sample at a first sample density.

FIG. 10B is a graph of an example Raman signal obtained by the Raman probe of FIG. 10A.

FIG. 11A is a side view of the example Raman probe and optical attachment of FIG. 4 submerged in a sample at a second sample density.

FIG. 11B is a graph of an example Raman signal obtained by the Raman probe of FIG. 11A.

FIG. 12 is a block diagram of an example method performed by the spectroscopy system of FIG. 1.

FIG. 13 is a block diagram of another example method performed by the spectroscopy system of FIG. 1.

While the present technology is susceptible to various modifications and alternative forms, specific aspects have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

Unless otherwise defined, all technical and scientific terms used herein have the same meaning as commonly understood by one of ordinary skill in the art. In case of conflict, the present document, including definitions, will control. Example methods and systems are described below, although methods and systems similar or equivalent to those described herein can be used in practice or testing of the present disclosure. The systems, methods, and examples disclosed herein are illustrative only and not intended to be limiting.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure is now described with reference to the drawings, wherein like reference numerals are used to refer to like elements throughout. In the following description, for purposes of explanation, numbers of specific details are set forth in order to provide an improved understanding of the present disclosure. It may be evident, however, that the systems and methods of the present disclosure may be practiced without one or more of these specific details. In other instances, well-known structures and devices are shown in block diagram form in order to facilitate describing the systems and methods of the present disclosure.

Optical analysis of a fluid may be performed in-situ by inserting an optical probe device into a sample (e.g., via an insertion port plumbed into the fluidic system of interest, into a vial or sample vessel, or the like) or by transporting a sample of the fluid to a flow cell of an optical analysis instrument, such as, for example, an optical probe that may be fixedly disposed relative to the flow cell or removably disposed relative to the flow cell. The optical probe can be used with various types of spectroscopy, such as, for example, Raman spectroscopy.

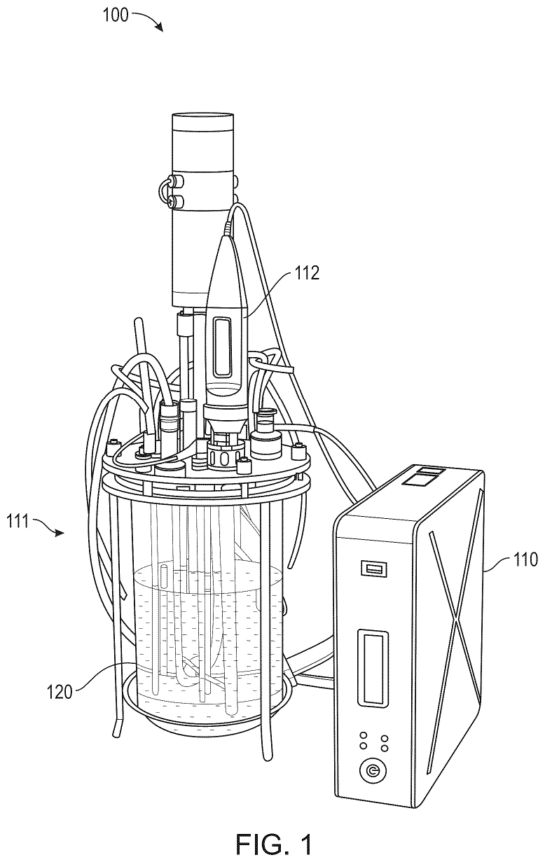

FIG. 1 is a perspective view of an example optical analysis system 100 in accordance with some aspects of the present disclosure. The optical analysis system 100 may be used to perform optical spectroscopy, such as, for example, Raman spectroscopy. As illustrated in FIG. 1, the optical analysis system 100 includes a controller component 110 and a probe component 112. A portion of the probe component 112 (for example, a probe head) is situated within a sample holder 111 such that the probe component 112 is positioned to project light onto a sample 120, as described below in more detail. The sample holder 111 may be, for example, a vat, a tank, a bioreactor, or some other storage unit for storing the sample 120. Other means of situating the probe component 112 to interact with a sample 120 may also be contemplated, as described herein.

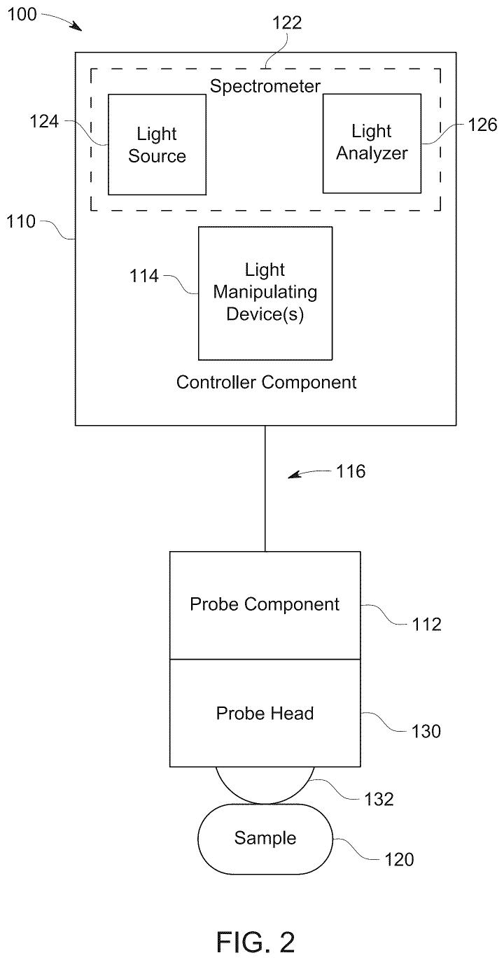

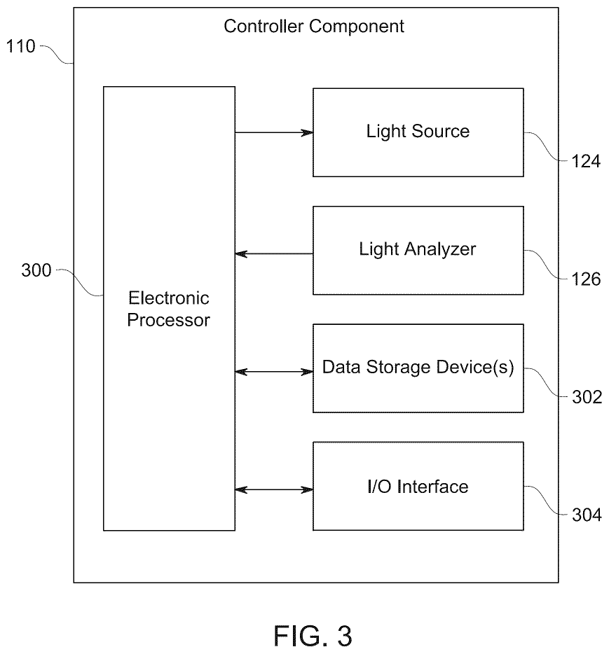

FIG. 2 is a schematic illustration of the example optical analysis system 100 of FIG. 1 in accordance with some aspects of the subject disclosure. The controller component 110 may include a spectrometer 122 including a light source 124 (e.g., a laser light source) and a light analyzer 126. The controller component 110 may also include one or more light manipulating devices 114 configured to receive laser light from the light source 124 and direct the laser light (e.g., finely focused) to the probe component 112. The controller component 110 may also include an electronic processor 300 and data storage device 302, as described with respect to FIG. 3.

The probe component 112 may be coupled to the controller component 110 via a fiber optic assembly 116 or other suitable light pipe. The light manipulating devices 114 are further configured to receive a Raman signal light from the fiber optic assembly 116 and direct the Raman signal light to the light analyzer 126. The light manipulating devices 114 may include one or more filters, reflectors, lens, or a combination thereof. For example, in some examples, a notch filter may direct laser light from the light source 124 toward a reflector that directs the laser light to a focusing lens that focuses and directs the laser light to a proximate end (i.e., proximate from a surface of the probe component 112 interfacing with the sample 120) of the fiber optic assembly 116. The directed laser light travels through the fiber optic assembly 116 to a distal end of the fiber optic assembly 116 positioned at a distal end of the probe component 112. In some examples, the light manipulating devices 114 include a collimating lens configured to direct Raman signal light received at the proximate end of the fiber optic assembly 116 through one or more filters (e.g., the notch filter) and to the light analyzer 126. The light analyzer 126 may include one or more detectors for detecting the intensity and the wavelength of the light. The detectors may output electronic signals reflecting the intensity or spectrum of the collected light. The optical analysis system 100 may further include one or more electronic processors and memory for storing computer readable instructions. By executing the computer readable instructions by the one or more electronic processors, the light analyzer 126 may process the output of the detector and generate compositional information of the sample. The optical analysis system 100 may also display the results to the operator via a display. The one or more electronic processors of the optical analysis system 100 may also be configured to implement the methods disclosed herein.

It should be understood that examples described herein may be used with various types of spectroscopy systems, including various types and configurations of Raman spectroscopy systems and is not limited to any specific configuration or arrangement of light manipulating devices. In other words, any suitable arrangement of light manipulating devices could be used to direct excitation laser light to the fiber optic assembly, receive Raman signal light from the fiber optic assembly, and direct the received Raman signal light to the light analyzer 126 of the spectrometer 122.

In some examples, the probe component 112 (including at least a portion of the fiber optic assembly 116) is part of, or integrated with, the controller component 110. For example, the probe component 112 may be mechanically attached to a housing that houses the controller component 110. In some implementations, the probe component 112 is retractable, in whole or in part, allowing the optical analysis system 100 to be more compact in a probe-retracted configuration than in a probe-extended configuration. As an example, in a hand-held implementation, a housing of the optical analysis system 100 can house the probe component 112 in a retracted position such that the probe component 112 can be extended, e.g., in a stiletto knife manner, rotated or folded out into an extended position, detached from the retracted position and reattached in an extended position, or other similar adjustable positions.

Also, in some examples, the probe component 112 can be flexibly connected to the controller component 110. For example, the probe component 112 may include a “goose-neck” type flexible portion, a pivot portion, a rotatable portion, or the like that allows the disposition of the probe component 112 relative to controller component 110 to be changed. For example, the probe component 112 may include a flexible portion, such as an elastomeric portion, to enable the probe component 112 to be employed in different positions relative to the controller component 110.

In some examples, the probe component 112 may be fixedly coupled with the controller component 110, may be integrated with the controller component 110, or may be removably coupled with the controller component 110. For example, in some examples, the controller component 110 includes one or more interfaces (e.g., integrated into a housing of the controller component 110) for receiving a probe component 112, such as, for example, using a snap-on, clamp-on, screw-on, slide-and-lock-on, or other type of coupling arrangement. Such interfaces may allow probe components 112 to be exchanged by “unplugging” one component and replacing it with a different component, which may be the same type of a probe component 112 or a different type of a probe component 112.

The probe component 112 includes a probe head 130 such as, for example, a probe head 130 enabling Raman spectrometry of an environment, such as, for example, for in-situ Raman spectrometry. Excitation laser light directed into the proximate end of the fiber optic assembly 116 exits the distal end of the fiber optic assembly 116 at an optical lens 132 of the probe head 130, such as the distal end. The probe head 130 may include a housing providing shielding and protecting the (portion of the) fiber optic assembly 116 positioned within the probe head 130. The housing of the probe head 130 may be sealed with the optical lens 132 to protect the fiber optic assembly 116 included in the probe head 130. In some examples, the optical lens 132 includes a spherical optical lens that serves as both an optical and sample interface. The spherical lens can be used as a sampling interface for the analysis of many types of samples, such as, for example, solids, powders, slurries, suspensions, particles, vapors, liquids, and the like. The samples may be homogenous, heterogenous, or comprised of multiple phases.

Excitation light (e.g., laser light) that is projected by the optical lens 132 onto the sample 120 causes the sample 120, in reaction to the excitation light, to emit a Raman signal light. The excitation light focuses on a focal region. The focal region locates at the effective focal length of the probe. The effective focal length of the probe may be the effective focal length of the optical lens 132. A part of the light emitted and/or scattered from the sample 120 (also referred to as Raman shifted light or emitted light or Raman signal light) travels back through the same conical configuration (i.e., the same light path) to the probe head 130 where the original excitation light was emitted from. A part of the Raman signal is reflected back to the probe head 130 by the reflective surface. The focal region of the excitation light overlaps with the focal region of the reflective surface. As such, both the frontscattered and backscattered Raman signal light is directed toward the light analyzer 126. In other words, light emitted from the probe head 130 initiates an analytical interrogation of the sample 120 (e.g., exciting atomic bonds of molecules in the sample 120) such that a Raman spectrum can be captured by the light analyzer 126 (e.g., including one or more detectors, such as, for example, one or more charged-coupled device (CCD) detectors) as a response to the interrogation of the sample 120. The Raman spectrum can be analyzed, for example, by the light analyzer 126 or by a separate component included in the controller component 110 or remote from the controller component 110. For example, the analysis of the Raman spectrum can be based on reference Raman spectra stored at controller component 110 or remote to but accessible to the controller component 110.

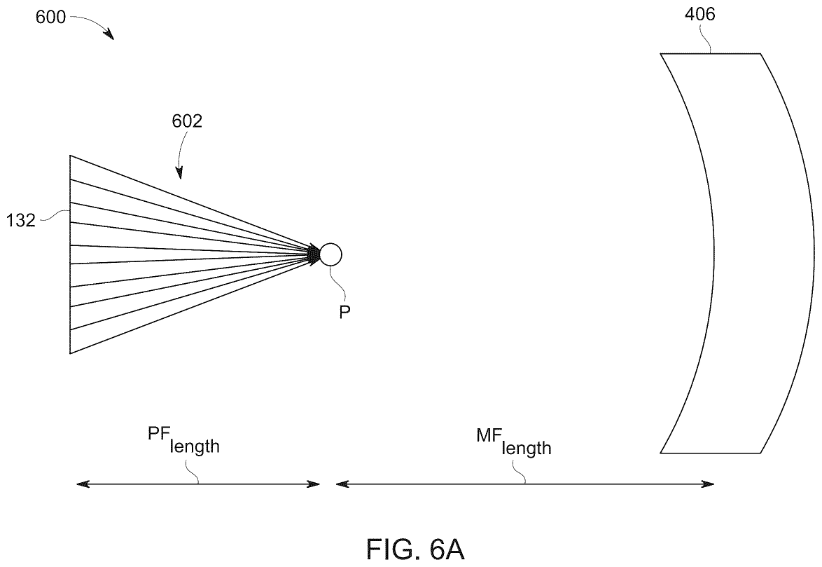

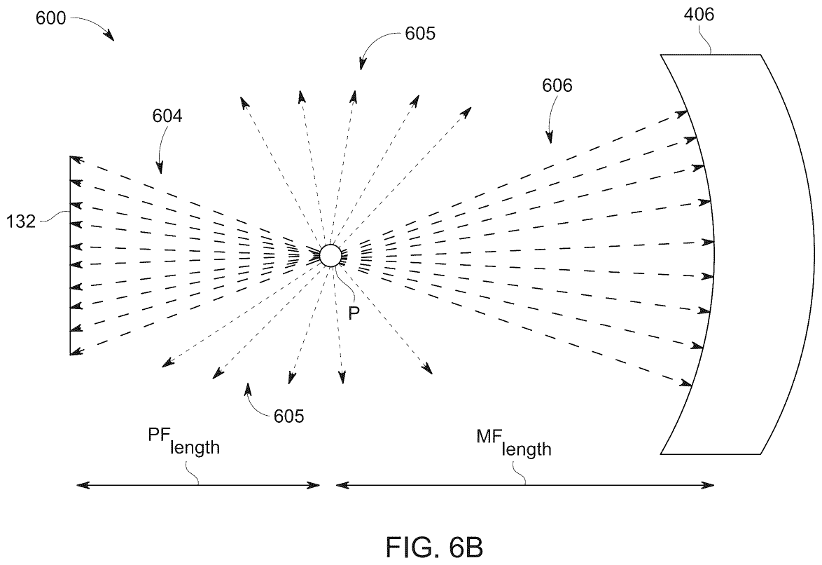

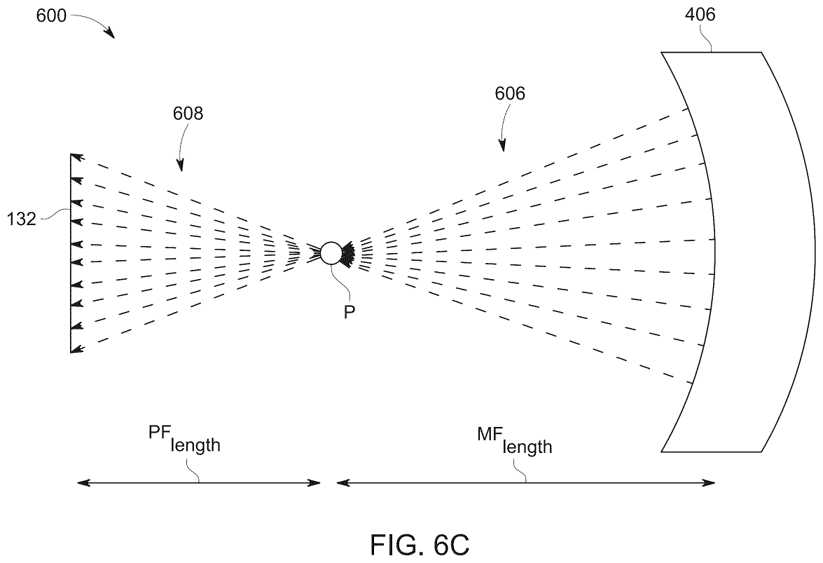

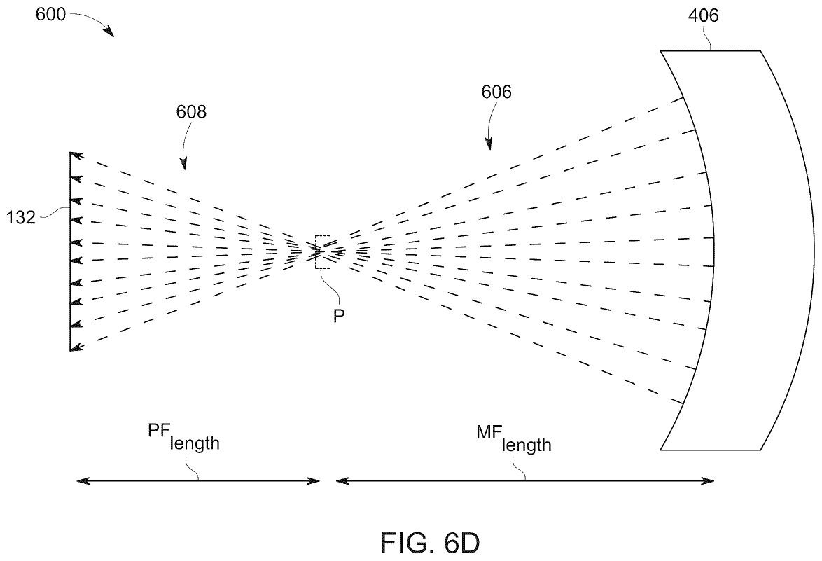

The Raman signal light that returns back to the probe head 130 from the focal region P (shown in FIG. 6A to FIG. 6B) is referred to herein as backscattered light. Some excitation light may also reflect off the sample 120 at the focal region P and return back to the probe head 130. Both the excitation light and the Raman signal light at the focal region P, however, also scatter forward (e.g., generally away from the probe head 130) and this light is referred to herein as frontscattered light.

It should be understood that various configurations and arrangements of probe components 112 may be used with the examples described herein and various suitable probe components 112, or various configurations of probe components 112, may exist that emit excitation light and receive reflected light (e.g., scattered light) from a sample.

Referring now to FIG. 3, the controller component 110 may include an electronic processor 300, data storage device(s) 302, and an input/output (I/O) interface 304, in addition to the light source 124 and the light analyzer 126 that form the spectrometer 122. However, it should be understood that the controller component 110 may have additional or fewer components. One or more of the light source 124, the light analyzer 126, the data storage device 302, and the interface 304 may be electronically connected with and controlled by the electronic processor 300. For example, the electronic processor 300 may receive data from the light analyzer 126 and identify the sample or determine the sample composition based on the received data.

The controller component 110 is suitable for the application and setting, and can include, for example, multiple electronic processors, multiple I/O interfaces, multiple data storage devices, or combinations thereof. In some implementations, some or all of the components included in the controller component 110 may be attached to one or more mother boards and enclosed in a housing (e.g., including plastic, metal and/or other materials). In some implementations, some of these components may be fabricated onto a single system-on-a-chip, or SoC (e.g., an SoC may include one or more processing devices and one or more storage devices). Additionally, one or more of these components may be situated in a separate housing. For example, the electronic processor 300 may be situated in a first housing, while the data storage device(s) 302 are situated in a second housing communicatively coupled to the first housing.

As used herein, “processors” or “electronic processor” refers to any device(s) or portion(s) of a device that process electronic data from registers and/or memory to transform that electronic data that may be stored in registers and/or memory. The electronic processor 300 may include one or more digital signal processors (DSPs), application-specific integrated circuits (ASICs), central processing units (CPUs), graphics processing units (GPUs), cryptoprocessors (specialized processors that execute cryptographic algorithms within hardware), server processors, or any other suitable processing devices.

The data storage device 302 may include one or more local or remote memory devices such as random-access memory (RAM) devices (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some implementations, the data storage device 302 may include memory that shares a die with a processor. In such an implementation, the memory may be used as a cache memory and may include embedded dynamic random-access memory (eDRAM) or spin transfer torque magnetic random-access memory (STT-MRAM), for example. In some implementations, the data storage device 302 may include non-transitory computer readable media having instructions thereon that, when executed by one or more processors (e.g., the electronic processor 300), causes the controller component 110 to store various applications and data for performing one or more of the methods described herein or portions described herein. It should be understood that each method described herein may be implemented via one application or multiple applications and, in some examples, the data storage device 302 stores additional data in various configurations.

The I/O interface 304 of controller component 110 may include one or more communication chips, connectors, and/or other hardware and software to govern communications between the controller component 110 and other components. For example, the I/O interface 304 may include circuitry for managing wireless communications for the transfer of data to and from the controller component 110. In some implementations, the I/O interface 304 may include one or more antennas (e.g., one or more antenna arrays) for receipt and/or transmission of wire communications.

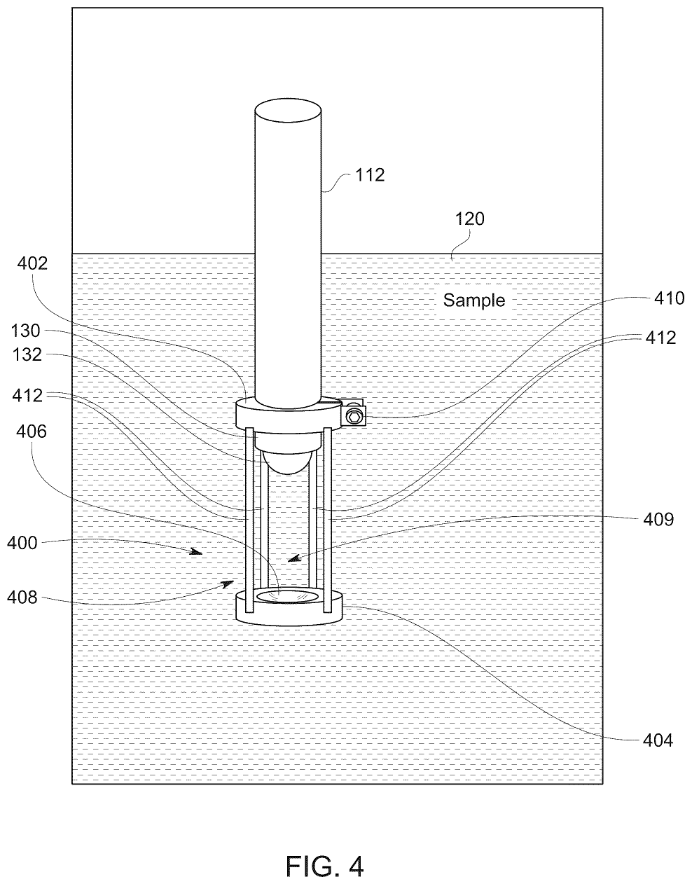

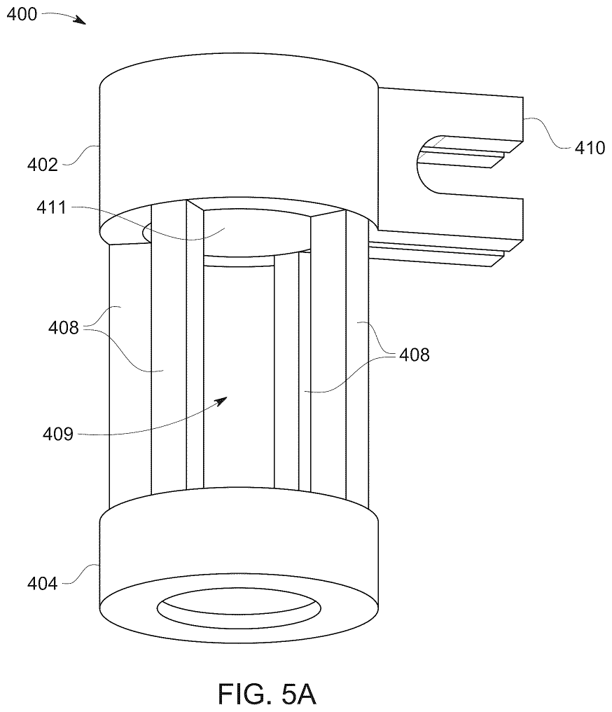

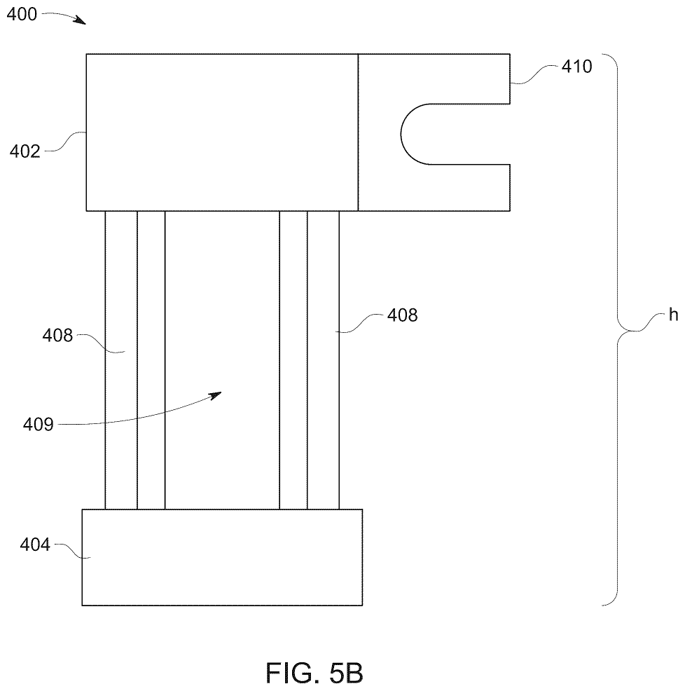

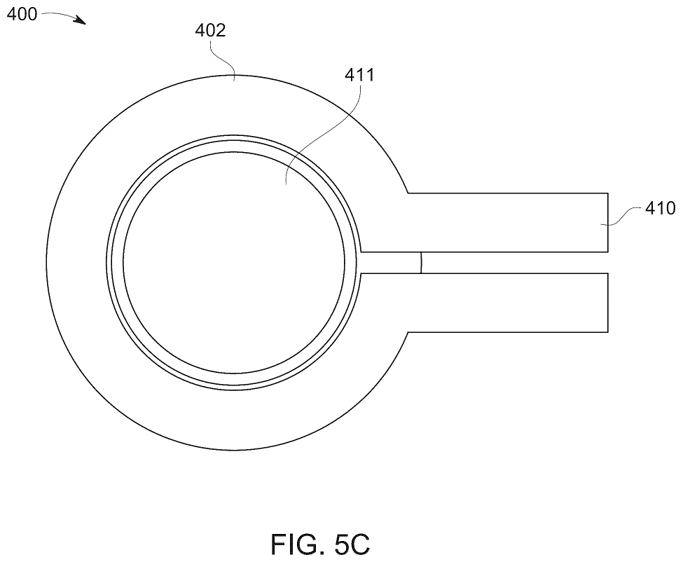

FIG. 4 is a side view of an example Raman probe (e.g., a probe component 112) with the attachment. The attachment is submerged in the sample 120. In the example of FIG. 4, the probe component 112 includes the probe head 130 and the optical lens 132 submerged in a sample 120. Sample 120 may be aqueous or powder. An attachment 400 is removably coupled to the probe head 130 (e.g., the distal end of the probe component 112). A perspective view of the attachment 400 is shown in FIG. 5A. A side view of the attachment 400 is shown in FIG. 5B. A top view of the optical attachment 400 is shown in FIG. 5C. The optical attachment 400 includes an attaching portion 402 (e.g., a mounting bracket), a holder 404 connected to the attaching portion 402 via a support structure 408, and a fastening portion 410.

The attaching portion 402 couples the optical attachment 400 to the probe component 112 at approximately the location of the probe head 130. In some instances, the attaching portion 402 includes a channel 411 in which the probe head 130 slides into the attaching portion 402, such that the attaching portion 402 surrounds a portion of the housing of the probe component 112. The attaching portion 402 may be sized such that a diameter of the channel 411 is approximately equal to the diameter of the probe head 130. In this instance, the attaching portion 402 may be friction fit such that friction maintains the position of the attaching portion 402 on the probe head 130, and the fastening portion 410 may be omitted. In other implementations, the diameter of the channel 411 may be larger than the diameter of the probe head 130. In such an implementation, the fastening portion 410 is provided to secure the attaching portion 402 to the probe head 130. For example, once the attaching portion 402 is positioned on the probe head 130, the fastening portion 410 includes a fastener (for example, a screw) that is tightened to secure the attaching portion 402 to the probe head 130.

The holder 404 supports a reflective surface 406 (e.g., a mirror). The reflective surface 406 is positioned from a side opposite to a side of the sample 120 being irradiated by the excitation light. When the optical lens 132 projects light onto the sample 120 to generate a Raman signal, the reflective surface 406 reflects frontscattered light back toward the optical lens 132. For example, FIG. 6A, FIG. 6B, and FIG. 6C illustrate an optical system 600 that includes the optical lens 132 and the reflective surface 406. FIG. 6A, FIG. 6B, and FIG. 6C show different steps of collecting a Raman signal light from the sample 120. For example, FIG. 6A shows the optical lens 132 projecting excitation light 602 (e.g., laser light) onto a sample 120 at focal region P. In FIG. 6A, excitation light 602 is shown focused at focal region P, located at an effective focal length PF_{length} (i.e., effective focal length of the probe) from the optical lens 312. The reflective surface 406 also reflects excitation light 602 back and cause an increase in the received signal. FIG. 6B illustrates the Raman light emission responsive to the excitation. The Raman light emission includes frontscattered light 606, backscattered light 604, and escaping light 605. For example, in some embodiments, the emitted Raman signal travels (e.g., uniformly) in all directions (e.g., three-dimensionally) from the focal region P. FIG. 6C illustrates the reflection of frontscattered light 606 and the resulting boosted Raman signal 608 (including the backscattered light (cone) 604) and the reflected frontscattered light 606). Accordingly, the reflective surface 406 reduces the amount of Raman signal that escapes detection by the probe by redirecting the frontscattered light 606 back toward the probe. For sake of clarity, the support structure 408 is omitted from FIG. 6A to FIG. 6C. The optical lens 132 is separated from the focal region P at the effective focal length PF_{length} (e.g., a probe effective focal length). The probe effective focal length PF_{length} may be, for example, between 1 mm and 8 mm, between 2 mm and 8 mm, between 4 mm and 8 mm, and the like. The focal region P is also separated from the reflective surface 406 by an effective focal length MF_{length} (e.g., the effective focal length of the reflective surface or mirror). The effective focal length of the reflective surface/mirror MF_{length} may be, for example, approximately 10 mm, approximately 12 mm, approximately 15 mm, or the like. Additionally, in some implementations, the diameter of the optical lens 132 is less than or equal to the total height h (shown in FIG. 5B) of the attachment 400. In other implementations, the diameter of the reflective surface 406 is less than or equal to the total height h of the optical attachment 400.

First, excitation light 602 (e.g., laser light), shown as a solid line, is projected by the optical lens 132 onto sample 120 at the focal region P, shown by FIG. 6A. Backscattered light 604 (shown as a dashed line representing an emitted Raman light signal) returns back to the probe head 130 from the focal region P, shown in FIG. 6B. Additionally, when light is projected by the optical lens 132 onto sample 120 at the focal region P, frontscattered light 606 (also shown as a dashed line representing an emitted Raman light signal) is scattered toward the reflective surface 406, shown in FIG. 6B. A portion of the light emitted by the sample 120, referred to herein as escaped light 605 (also shown as a dotted line representing an emitted Raman light signal), is lost and is not returned to the optical lens 132. While FIG. 6B only shows a few illustrative examples of the escaped light 605, one skilled in the art will appreciate that the sample 120 may emit light homogenously in all directions (three-dimensionally) from the focal region P, including directions perpendicular to the direction of the backscattered light 604 and the frontscattered light 606. The reflective surface 406 reflects the frontscattered light 606 back to the optical lens 132 through the focal region P, shown in FIG. 6C. In this manner, the boosted Raman signal 608 received by the optical lens 132 includes both the frontscattered light 606 (a cone of emitted light signal) and the backscattered light 604 (as a cone of emitted light signal). The optical lens 132 may collimate the received boosted Raman signal 608 for transport in the fiber optic assembly 116. The angle of the light 602 and the frontscattered light 606 is dependent on the value of the probe focal length PF_{length}.

In some embodiments, to direct the frontscattered light 606 along the same optical path as the backscattered light 604, the reflective surface 406 has a curved surface. For example, reflective surface 406 may be a concave mirror, a spherical mirror, a parabolic mirror, or the like. Additionally, a diameter of the reflective surface 406 may be at least equal to, or greater than, a diameter of the optical lens 132. The reflective surface 406 may have a focal region that overlaps with the focal region of the probe optics, which may be impacted by or equivalent to, for example, the optical lens 132.

FIG. 6A to FIG. 6C illustrate a particular example of the reflective surface 406 as a concave spherical mirror or a parabolic mirror. However, as noted above, other shapes of the reflective surface 406 may also be implemented. For example, a plurality of flat mirrors may be arranged in a curved (e.g., a parabolic) manner such that light is reflected through the focal region P of the optical lens 132 and back to the optical lens 132.

While the focal region P may appear to be a single point in FIG. 6A to FIG. 6C at which the light 602 reflects from and the reflected frontscattered light 606 passes through, in practice the focal region P may be a region through which the reflected frontscattered light 606 passes, shown in FIG. 6D.

Returning to FIGS. 4 and 5, the support structure 408 connects the attaching portion 402 to the holder 404. The support structure 408 may include, for example, legs, beams, prongs, tines, or the like that connect the attaching portion 402 and the holder 404. In the example shown in FIGS. 4 and 5, the support structure 408 is defined by a plurality of support members 412 extending between the attaching portion 402 and the holder 404. More specifically, four support members 412, or legs, are illustrated. However, fewer or more legs may be provided. Each support member 412 is spaced apart from an adjacent support member 412. As such, each adjacent pair of support members 412 partially defines an aperture 409 (or an opening 409). Stated another way, each aperture 409 is defined on opposing sides by a pair of the support members 412. Accordingly, the support structure 408 illustrated in FIG. 4 (e.g., via the plurality of support members 412) defines a plurality of apertures 409. The support members 412 positions the holder 404 at a distance from the attaching portion 402. The plurality of apertures 409 facilitate unobstructed flow (or passage) of sample 120 through the optical attachment 400. The plurality of apertures 409 can be referred to as flow apertures 409 and can facilitate a free flow (or a turbulent flow) of the sample 120 through the optical attachment 400, and more specifically through an area between the optical lens 132 and the reflective surface 406. In the illustrated configuration, the plurality of apertures 409 are arranged such that pairs of the plurality of apertures 409 are configured to receive and retain linearly aligned a vessel containing the sample 120. For example, each pair of apertures 409 can be horizontally aligned.

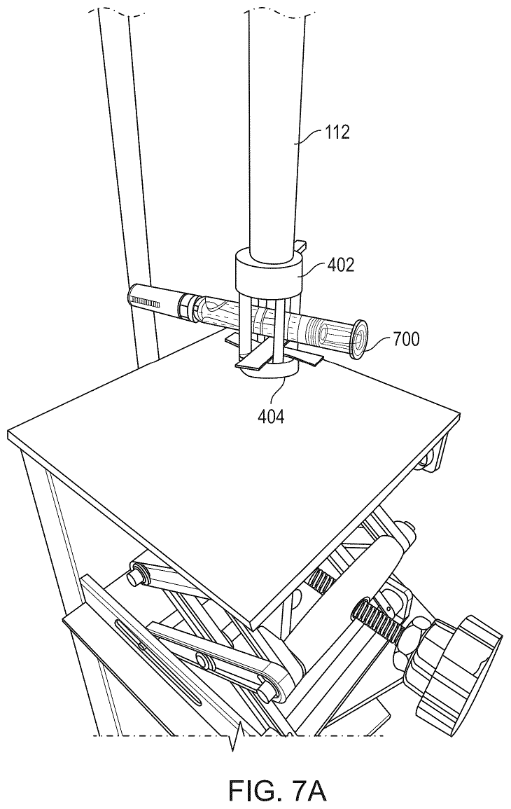

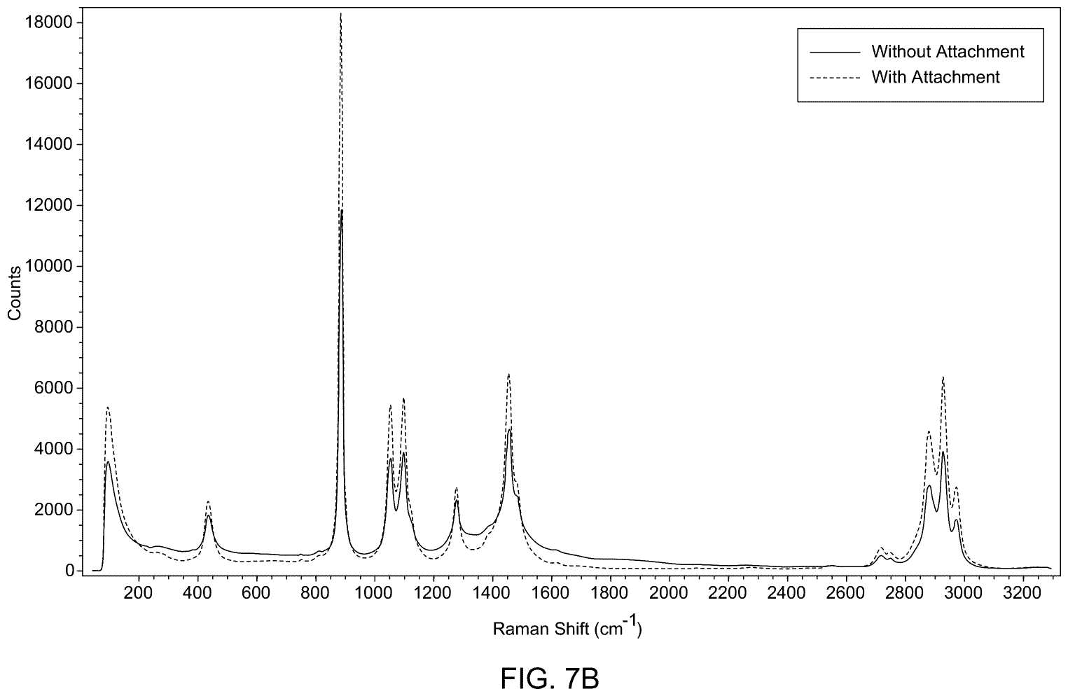

In some implementations, rather than being submerged in a liquid sample 120, the optical attachment 400 is configured to receive a vial or other sample holder between the attaching portion 402 and the holder 404. For example, FIG. 7A illustrates one example of the optical attachment 400 receiving a vial 700 (e.g., a vessel) filled with a sample 120. The vial 700 may be situated such that the sample 120 stored within the vial 700 is situated at the focal region P (not shown in FIG. 7A). The vial 700 is received and retained by a pair of aligned apertures 409 (shown in FIG. 4). In some implementations, the sample 120 is secured to the optical attachment 400 in a manner that does not impede the travel of the backscattered light and the frontscattered light.

FIG. 7B shows the Raman signal received from a sample comprising ethanol and monoclonal antibodies held in the vial 700 using the probe component 112 with the attachment 400 (as shown in FIG. 7A) and without the attachment. With the attachment attached, the boosted Raman signal measured was at least 50% greater than the Raman signal measured without the attachment.

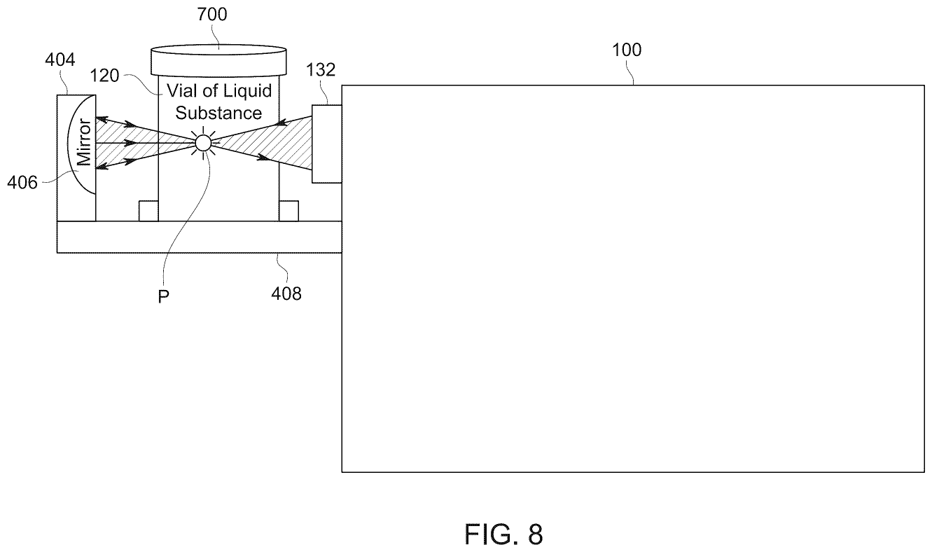

FIG. 8 illustrates another example of the optical attachment 400 receiving a vial 700. In the example of FIG. 8, light from optical analysis system 100 is delivered to sample 120 within the vial 700 via optical lens 132. The vial 700 is supported by the support structure 408 such that the effective focal point P of the excitation light from the optical analysis system 100 is within the sample 120. Frontscattered light is reflected back by the reflective surface 406 held by holder 404. In this example, an attaching portion of the optical attachment 400 may be couplable (e.g., removably) to a housing of the optical analysis system 100.

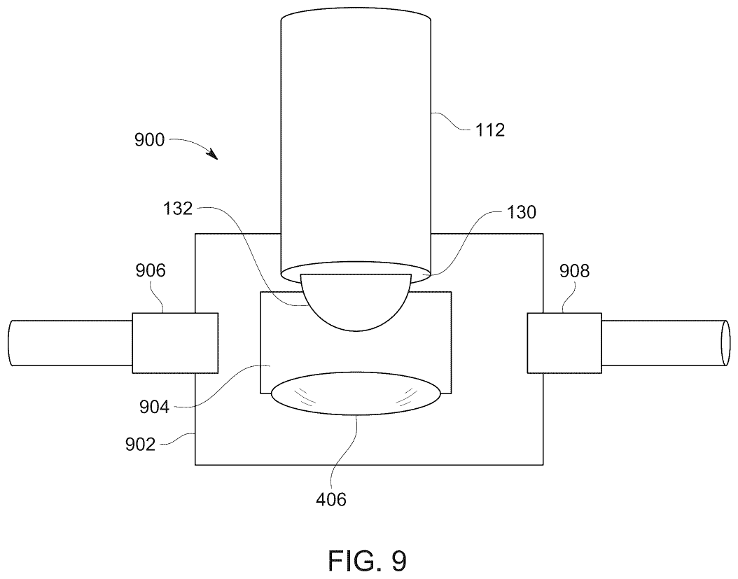

In yet another example, rather than the support structure 408 including a plurality of legs, the optical attachment 400 may include an enclosed housing. FIG. 9 illustrates an example optical attachment 900 coupled to the probe component 112. The optical attachment 900 includes a housing 902 that forms a chamber 904 in which a sample 120 (not shown) may be situated. The housing is coupled to the probe at the attaching portion. The wall of the housing serves as the support structure that extends from the probe to the reflective surface. The bottom of the housing holds the reflective surface. The housing 902 supports reflective surface 406 such that the reflective surface 406 faces the optical lens 132 of the probe component 112.

The housing 902 is configured to receive the probe head 130 (allowing the optical lens 132 to at least partially enter the chamber 904). The housing 902 includes apertures that function as a fluid inlet 906 and a fluid outlet 908. Fluid sample 120 may travel through the fluid inlet 906 to enter the chamber 904 and exit the chamber 904 through the fluid outlet 908 such that constant flow of the sample 120 is provided. Raman signal is received by the probe component 112 as previously described.

While some examples described herein refer to the optical attachment 400 as a separate accessory configured to be removably coupled to the probe component 112 or other portions of the optical analysis system 100, in other examples, the optical attachment 400 can be formed with the probe component 112 or other portions of the optical analysis system 100 such that the optical attachment 400 is not configured to be removed. As a non-limiting example, the holder 404 carrying the reflective surface 406 can be fastened to the probe component 112, such as to an outer housing of the probe component 112. In this example, the support structure 408 can be welded, bonded, fused or otherwise fastened to the outer housing of the probe component 112 such that the components are not removable.

Accordingly, examples described herein provide for receiving both backscattered light and frontscattered light, thereby receiving a boosted Raman signal. Improved signal quality enables users of the optical analysis system 100 to obtain more accurate and reliable data for monitoring parameters of the sample 120. For example, the boosted Raman signal allows for a more precise measurement of various biomolecules and metabolites within the sample holder 111.

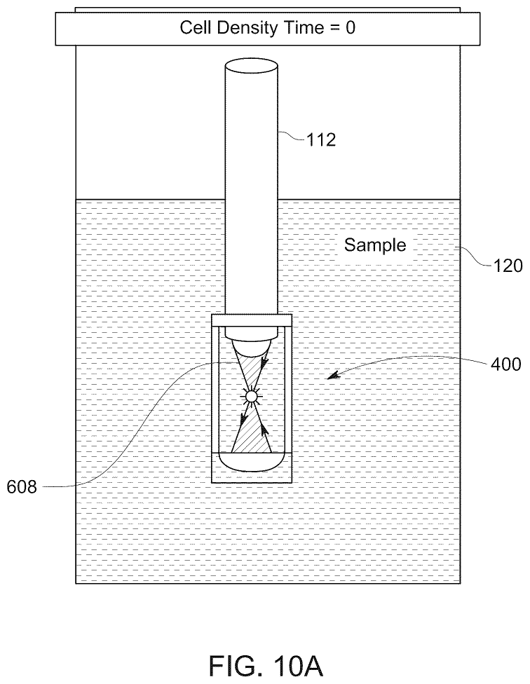

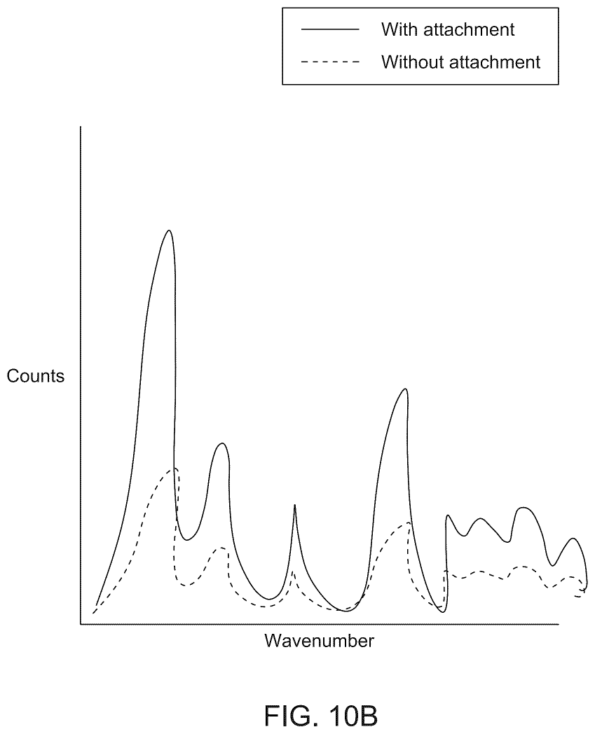

In some instances, the boosted Raman signal allows the controller component 110 to monitor changes in the sample 120 over time. For example, cell density may change within the sample 120 over time. For example, FIG. 10A illustrates the probe component 112 interrogating a sample 120 in a bioreactor at an initial time (t=0). The sample 120 is composed of cells in the cell media that initially has a low density. The boosted Raman signal 608 generated by the optical attachment 400 may approximately be twice the strength of the Raman signal light that would be received by the probe component 112 without the optical attachment 400, shown in FIG. 10B.

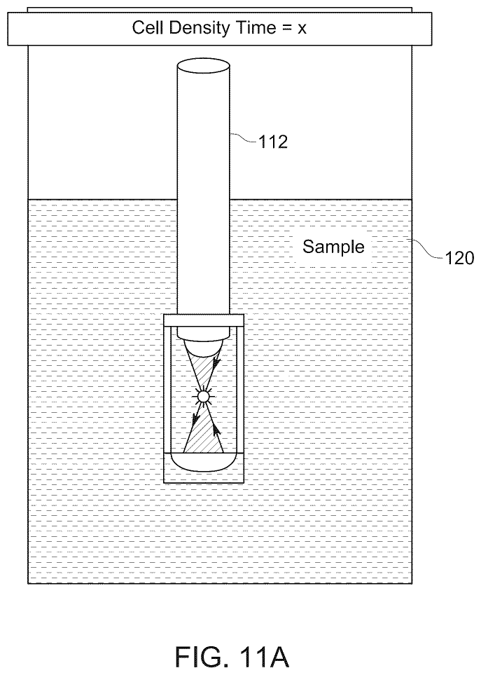

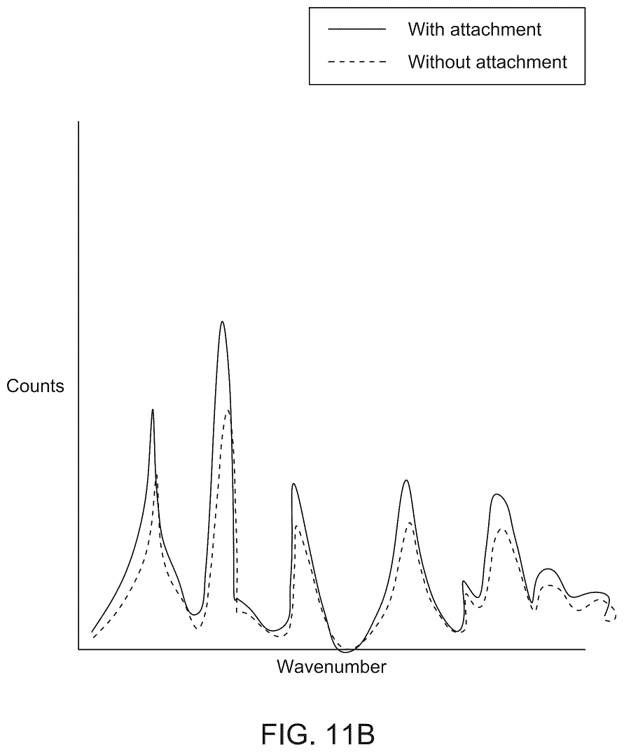

However, over time during the interrogation of the sample 120, the density of the cell media may increase as the cells multiply. FIG. 11A illustrates the probe component 112 interrogating the sample 120 at a future time (t=x), where x occurs after the initial time referenced in FIG. 10A. As the cell density increases, optical density may also increase and more of the scattered light may be absorbed by the sample 120, interfering with the capability of the frontscattered light returning to the optical lens 132. Accordingly, the amplitude of the boosted Raman signal 608 may drop significantly and may be approximately equal to the strength of the Raman signal light that would be received by the probe component 112 without the optical attachment 400, shown in FIG. 11B.

As the magnitude of the boosted Raman signal 608 is dependent on the cell density, in some instances, the controller component 110 may estimate the cell density of the sample 120 based on the magnitude of the boosted Raman signal 608. For example, the data storage device 302 may store a model that relates the cell density to the magnitude of the boosted Raman signal 608. The magnitude of the boosted Raman signal 608 received by the light analyzer 126 is compared by the electronic processor 300 to the model to determine the cell density. The model may include, for example, a machine learning model, a table storing values of magnitudes of the boosted Raman signal light 608 and cell densities, a function relating the magnitude of the boosted Raman signal light 608 and cell density, and the like.

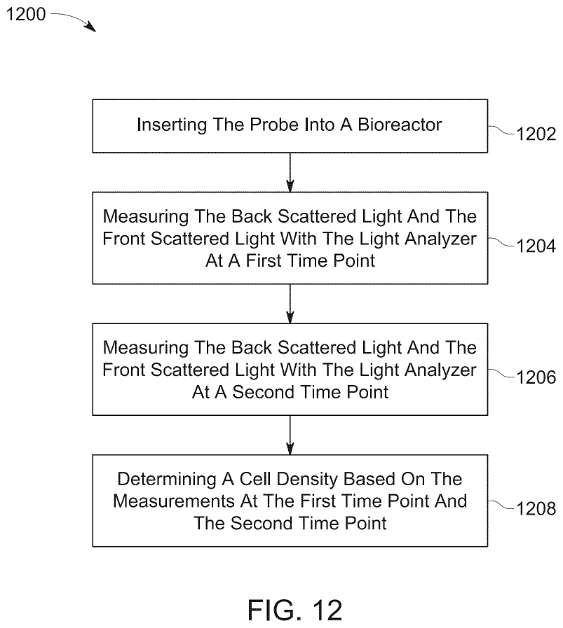

For example, the boosted Raman signal 608 may be used by the optical analysis system 100 to monitor changes in a sample 120 in a bioreactor over time. FIG. 12 illustrates a block diagram of an example method 1200 for monitoring changes in a sample 120. The method 1200 may be performed by the controller component 110 (e.g., the electronic processor 300). The steps of the method 1200 are described in an iterative manner for descriptive purposes. Various steps described herein with respect to the method 1200 are capable of being executed simultaneously, in parallel, or in an order that differs from the illustrated serial and iterative manner of execution.

At block 1202, the method 1200 includes inserting the probe into a bioreactor. For example, the probe component 112 of the optical analysis system 100 (e.g., a probe head) as well as the attachment may be inserted into the bioreactor.

At block 1204, the method 1200 includes measuring the backscattered light and the frontscattered light with the light analyzer 126 at a first time point. At block 1206, the method 1200 includes measuring the backscattered light and the frontscattered light at a second time point. The second time point may be a point in time after the first time point (i.e., the second time point may be later than the first time point).

At block 1208, the method 1200 includes determining a cell density based on the measurements at the first time point and the second time point. For example, the measurements at the first time point and the second point may be used by the controller component 110 (e.g., electronic processor 300) to determine (e.g., estimate) a cell density of the sample 120. As noted above, the electronic processor 300 may use the measurements from the two time points and a model (e.g., a machine learned model, a table, a function, or the like (e.g., a threshold)) to correlate a change in the boosted Raman signal light 608 (e.g., a difference between the measurement at the first time point and the measurement at the second time point) with a change in cell density occurring between the first time point and the second time point or a current cell density (i.e., a cell density at the second time point), which provides status information of the sample 120 within the bioreactor and can be used as feedback to control bioreactor operation. As one non-limiting example, when a difference between the measurement at the first time point and the second time point reaches a predetermined threshold, the cell density of the sample may be estimated to have reached a predetermined density level, which may be used to control operation of the bioreactor (e.g., modify or stop operation)

Further, characteristics of the boosted Raman signal 608 (for example, the magnitude, the frequency, and the like) may be used by the controller component 110 (or a separate controller) for determining a state of the optical attachment 400. For example, during operation of the probe component 112, certain components of the optical attachment 400 may experience wear and/or damage over time. As one example, the reflective surface 406 may become misaligned or smudged, impacting the quality of the boosted Raman signal 608 received by the optical lens 132. By monitoring changes of the boosted Raman signal 608 over time for a known sample 120, the controller component 110 may determine that the optical attachment 400 needs repair or should be replaced.

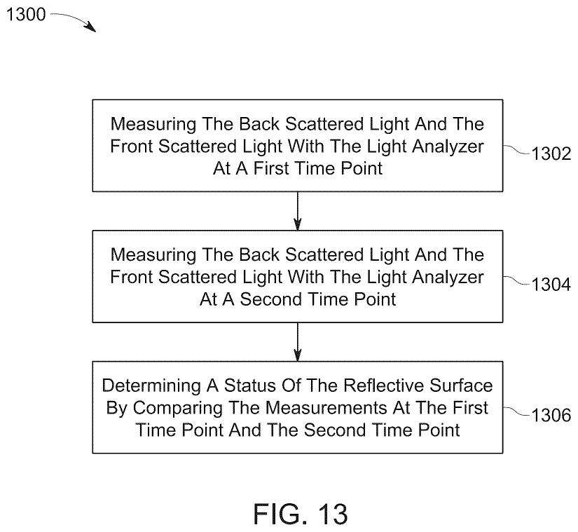

FIG. 13 illustrates a block diagram of an example method 1300 for determining a status of the reflective surface 406. The method 1300 may be performed by the controller component 110 (e.g., the electronic processor 300). The steps of the method 1300 are described in an iterative manner for descriptive purposes. Various steps described herein with respect to the method 1200 are capable of being executed simultaneously, in parallel, or in an order that differs from the illustrated serial and iterative manner of execution.

At block 1302, the method 1300 includes measuring the backscattered light and the frontscattered light with the light analyzer 126 at a first time point. At block 1304, the method 1300 includes measuring the backscattered light and the frontscattered light at a second time point. The second time point may be a point in time after the first time point (i.e., the second time point may be later than the first time point).

At block 1306, the method 1300 includes determining a status of the reflective surface by comparing the measurements at the first time point and the second time point. For example, a difference in the measurements may be compared to a predetermined threshold and, in response to the difference exceeding the predetermined threshold, the electronic processor 300 may trigger one or more maintenance activities to address a state of the optical attachment 400 (e.g., the reflective surface). As one non-limiting example, the electronic processor 300 may be configured to issue one or more maintenance alerts or orders in response to the difference between the measurements exceeding the predetermined threshold (within a predetermined amount of time), which the electronic processor 300 correlates with a degraded status of the optical attachment 400.

In addition to improved signal quality and measurement precision, the optical attachment 400 provides for non-invasive monitoring within a bioreactor, as additional Raman signal light is collected without the use of additional probes or physical intervention within the bioreactor. This non-invasive approach provides minimal disruption to the sample interrogation process and reduces the risk of contamination or interference with cell growth and product formation.

As, in some examples, the optical attachment 400 is removably attachable from the probe component 112, the optical attachment 400 is capable of being integrated with existing biopharmaceutical workflows. The versatility of the optical attachment 400 allows researchers to apply examples described herein to a wide range of bioreactor configurations and processes. Additionally, improved signal quality and measurement precision assists researchers in identifying and addressing process deviations or inefficiencies in real-time, enabling timely interventions and adjustments, shorter development cycles, and reduced manufacturing costs.

As described above in the detailed description, reference is made to the accompanying drawings that form a part hereof wherein like numerals designate like parts throughout, and in which is shown, by way of illustration, implementations that may be practiced. It is to be understood that other implementations may be utilized, and structured or logical changes may be made, without departing from the scope of the present disclosure. Therefore, the detailed description as described above is not to be taken in a limiting sense.

Various operations may be described as multiple discrete actions or operations in turn, in a manner that is most helpful in understanding the subject matter disclosed herein. However, the order of description should be construed as to imply that these operations are necessarily order dependent. In particular, these operations may not be performed in the order of presentation. Operations described may be performed in a different order from the described implementation. Various additional operations may be performed, and/or described operations may be omitted in additional implementations.

### Clauses

Implementations of the present disclosure are disclosed in the following clauses:

Clause 1. An attachment for an optical analysis system that emits an excitation light toward a sample, the attachment including: a reflective surface positioned to reflect light from the sample toward a probe of the optical analysis system; and a holder supporting the reflective surface, wherein the sample is positioned between the probe and the reflective surface.

Clause 2. The attachment of clause 1, wherein the reflective surface is positioned from a side opposite to a side of the sample being irradiated by the excitation light.

Clause 3. The attachment of clause 1 or 2, further comprising an attaching portion configured to be coupled to the probe and one or more support structures extending between the probe and the reflective surface.

Clause 4. The attachment of clause 3, wherein the attaching portion is removably couplable with the probe.

Clause 5. The attachment of clause 3, wherein the one or more support structures define a plurality of flow apertures for receiving the sample.

Clause 6. The attachment of any of clauses 1-5, further including an attaching portion configured to be coupled to a housing of the optical analysis system and one or more support structures extending between the attaching portion and the holder.

Clause 7. The attachment of clause 6, wherein the attaching portion is removably couplable with the housing of the optical analysis system.

Clause 8. The attachment of any of clauses 1-7, wherein the reflective surface includes a reflective surface of a mirror having a diameter at least as large as a diameter of the probe.

Clause 9. The attachment of any of clauses 1-8, wherein a focal region of the reflective surface overlaps with a focal region of the excitation light at the sample.

Clause 10. An optical analysis system, comprising: a light source generating an excitation light; a light analyzer; an attachment of any of clauses 1-9, and an optical lens configured to: direct the excitation light to a sample positioned between the optical lens and the reflective surface of the attachment; receive a backscattered light and a frontscattered light from the sample, wherein the frontscattered light is reflected from the reflective surface; and direct the received backscattered light and frontscattered light to the light analyzer.

Clause 11. The optical analysis system of clause 10, wherein the optical lens is included in the probe.

Clause 12. The optical analysis system of clause 11, wherein the holder is removably coupled with a housing of the probe.

Clause 13. The optical analysis system of clause 11, wherein the holder is removably coupled with a housing of the optical analysis system.

Clause 14. The optical analysis system of any of clauses 10-13, wherein the reflective surface is curved, wherein a focal region of the reflective surface overlaps with a focal region of the excitation light at the sample.

Clause 15. The optical analysis system of any of clauses 10-14, wherein the reflective surface reflects the frontscattered light along a same optical path as the backscattered light.

Clause 16. The optical analysis system of clause 15, further comprising an attaching portion configured to be coupled to the probe and one or more support structures extending between the probe and the reflective surface, wherein the one or more support structures define a plurality of flow apertures for receiving the sample, and wherein the plurality of flow apertures are configured to receive a vessel carrying the sample.

Clause 17. The optical analysis system of clause 15, wherein the reflective surface and at least a part of the holder is submerged in the sample while directing the excitation light to the sample.

Clause 18. The optical analysis system of any of clauses 10-17, further comprising computer readable instructions stored in a memory and a processor, by executing the computer readable instructions in the processor, the optical analysis system is configured to generate a spectrum of the light directed to the light analyzer.

Clause 19. A method of using the optical analysis system of any of clauses 10-18, for monitoring the sample in a bioreactor, including: inserting the probe into the bioreactor; measuring the backscattered light and the frontscattered light with the light analyzer at a first time point; measuring the backscattered light and the frontscattered light with the light analyzer at a later, second, time point; and determining a cell density based on the measurement at the first time point and the second time point.

Clause 20. A method of using the optical analysis system of any of clauses 10-18, including: measuring the backscattered light and the frontscattered light with the light analyzer at a first time point; measuring the backscattered light and the frontscattered light with the light analyzer at a later, second time point; and determining a status of the reflective surface by comparing the measurements at the first time point and the second time point.

What is claimed is:

1. An attachment for an optical analysis system that emits an excitation light toward a sample, the attachment including:
a reflective surface positioned to reflect light from the sample toward a probe of the optical analysis system; and
a holder supporting the reflective surface, wherein the sample is positioned between the probe and the reflective surface.

2. The attachment of claim 1, wherein the reflective surface is positioned from a side opposite to a side of the sample being irradiated by the excitation light.

3. The attachment of claim 1, further comprising an attaching portion configured to be coupled to the probe and one or more support structures extending between the probe and the reflective surface.

4. The attachment of claim 3, wherein the attaching portion is removably couplable with the probe.

5. The attachment of claim 3, wherein the one or more support structures define a plurality of flow apertures for receiving the sample.

6. The attachment of claim 1, further including an attaching portion configured to be coupled to a housing of the optical analysis system and one or more support structures extending between the attaching portion and the holder.

7. The attachment of claim 6, wherein the attaching portion is removably couplable with the housing of the optical analysis system.

8. The attachment of claim 1, wherein the reflective surface includes a reflective surface of a mirror having a diameter at least as large as a diameter of the probe.

9. The attachment of claim 1, wherein a focal region of the reflective surface overlaps with a focal region of the excitation light at the sample.

10. An optical analysis system, comprising:
a light source generating an excitation light;
a light analyzer;
an attachment including
a reflective surface positioned to reflect light from a sample toward a probe of the optical analysis system, and
a holder supporting the reflective surface, and
an optical lens configured to:
direct the excitation light to the sample positioned between the optical lens and the reflective surface of the attachment;
receive a backscattered light and a frontscattered light from the sample, wherein the frontscattered light is reflected from the reflective surface; and
direct the received backscattered light and frontscattered light to the light analyzer.

11. The optical analysis system of claim 10, wherein the optical lens is included in the probe.

12. The optical analysis system of claim 11, wherein the holder is removably coupled with a housing of the probe.

13. The optical analysis system of claim 11, wherein the holder is removably coupled with a housing of the optical analysis system.

14. The optical analysis system of claim 10, wherein the reflective surface is curved, wherein a focal region of the reflective surface overlaps with a focal region of the excitation light at the sample.

15. The optical analysis system of claim 10, wherein the reflective surface reflects the frontscattered light along a same optical path as the backscattered light.

16. The optical analysis system of claim 15, further comprising an attaching portion configured to be coupled to the probe and one or more support structures extending between the probe and the reflective surface, wherein the one or more support structures define a plurality of flow apertures for receiving the sample, and wherein the plurality of flow apertures are configured to receive a vessel carrying the sample.

17. The optical analysis system of claim 15, wherein the reflective surface and at least a part of the holder is submerged in the sample while directing the excitation light to the sample.

18. The optical analysis system of claim 10, further comprising computer readable instructions stored in a memory and a processor, by executing the computer readable instructions in the processor, the optical analysis system is configured to generate a spectrum of the light directed to the light analyzer.

19. The optical analysis system of claim 10, wherein the sample is in a bioreactor and further comprising computer readable instructions stored in a memory and a processor, by executing the computer readable instructions in the processor, the optical analysis system is configured to measure the backscattered light and the frontscattered light at a first time point and at a later, second, time point and determine a cell density based on the measurement at the first time point and the second time point.

20. The optical analysis system of claim 10, further comprising computer readable instructions stored in a memory and a processor, by executing the computer readable instructions in the processor, the optical analysis system is configured to measure the backscattered light and the frontscattered light at a first time point and at a later, second time point and determine a status of the reflective surface by comparing the measurements at the first time point and the second time point.
